(19) United States
(12) Patent Application Publication
(10) Pub. No.: US 2011/0052702 A1
(43) Pub. Date: Mar. 3, 2011

(54) Method and Apparatus for Producing Organic Nanotubes

(75) Inventors: Masumi Asakawa, Ibaraki (JP); Masaki Kogiso, Ibaraki (JP); Toshimi Shimizu, Ibaraki (JP)

(73) Assignee: National Institute of Advanced Industrial Science and Technology, Tokyo (JP)

(21) Appl. No.: 12/863,434

(22) PCT Filed: Jan. 21, 2009
(86) PCT No.: PCT/JP2009/050863

§ 371 Date: Jul. 16, 2010

(30) Foreign Application Priority Data
Jan. 24, 2008 (JP) 2008-013280

(51) Int. Cl. A61K 9/14; F28D 21/00; A61K 38/00; A61K 31/7028; A61P 43/00; B82Y 5/00

(52) U.S. Cl. 424/489; 422/208; 514/1.1; 514/42; 977/700; 977/788

(57) ABSTRACT

An object of the present invention is to provide a method and apparatus capable of continuously producing organic nanotubes, wherein an organic nanotube material dispersion solution consisting of an organic nanotube material and an organic solvent is pressurized and caused to pass through a very narrow orifice.

An apparatus for producing organic nanotubes according to the present invention includes: a tank that contains an organic nanotube material dispersion solution consisting of an organic nanotube material and an organic solvent; a pump that pressurizes the organic nanotube material dispersion solution from the tank so that the organic nanotube material dispersion solution is carried under high pressure; a cylindrical casing for continuously flowing the organic nanotube material dispersion solution carried from the pump under pressure; an orifice being placed in the cylindrical casing; an organic nanotube precipitation pipe being coupled to an outlet of the cylindrical casing; and drying means following the organic nanotube precipitation pipe.

### TECHNICAL FIELD

The present invention relates to a method and apparatus for producing organic nanotubes that are useful as a highly-functional material, such as an encapsulating/separating material or a sustained drug release material in the field of medical/chemical products, in a simple manner and in large quantities.

### BACKGROUND ART

Among nanotubular materials having a pore with a size of 0.5 to 500 nm, a carbon nanotube is an inorganic nanotube which was artificially synthesized for the first time. With expectations for features of the carbon nanotube, such as size, configuration and chemical structure, studies on applications to nanoscale electronic devices, high-strength materials, electron emission and gas storage, and researches on mass production techniques toward practical use thereof, have been strenuously carried out (see Patent Literature 1).

Cyclodextrin is known as an organic cyclic compound having a pore with a size of 1 nm or less, which is capable of encapsulating various low-molecular organic compounds in an annular hollow space thereof. Thus, through the research and development for contribution to the fields of health foods, cosmetic products, household antibacterial/deodorant products and industry/agriculture/environment, various cyclodextrin-encapsulated products have been commercialized (see, for example, Patent Literature 2). Cyclodextrin can be produced in large quantities, and biological safety thereof is ensured because its structure is made up of annularly linked 6 to 8 glucose units. Thus, a wide range of application is being studied.

The inventors of this application have already developed an organic nanotube obtainable through self-assembling in an aqueous solution (see, for example, Patent Literature 3 and Non-Patent Literatures 1 to 3). This organic nanotube developed by the inventors is a hollow cylinder having an inner pore with a size of 5 to 500 nm which is one digit greater than that of cyclodextrin. Thus, it has a potential to be able to capture a functional substance with a diameter of 5 to 500 nm that cannot be encapsulated in cyclodextrin, such as protein, virus, chemicals or metal nanoparticles, within the hollow cylinder, and therefore there are great expectations to utilize the features for various purposes.

The conventional organic nanotube has been synthesized in an aqueous solution. Thus, in a production process, it is necessary to use a large volume of water, and perform an operation of heating and stirring the solution and an operation of maintaining the solution in a stationary state for a long period of time, which causes difficulty in ensuring mass-productivity. Moreover, the organic nanotube synthesized in an aqueous solution strongly holds water in its structure (this organic nanotube will hereinafter be referred to as “water-containing organic nanotube”), and the water is hardly removed by conventional methods, which causes a problem of being unable to efficiently encapsulate a functional substance into the water-containing organic nanotube.
Patent Literature 1
Japanese Unexamined Patent Application Publication (Translation of PCT Application), Tokuhyo, No. 2003-535794 A
Patent Literature 2
Japanese Patent Application Publication, Tokukai, No. 2005-306763 A
Patent Literature 3
Japanese Patent No. 3664401
Non-Patent Literature 1
Langmuir, 2005, 21, 743
Non-Patent Literature 2
Chem. Comm., 2004, 500
Non-Patent Literature 3
Chem. Mater., 2004, 16, 2826

### SUMMARY OF INVENTION

### Technical Problem

Through various researches for solving the above problems, the inventors found that a water-free organic nanotube can be produced in a simple manner and in large quantities by allowing an N-glycoside type glycolipid or a peptide lipid to self-assemble in an organic solvent, instead of water. Based on this knowledge, the inventors have already filed patent applications for an organic nanotube and production method thereof (see PCT/JP2007/061703 and PCT/JP2007/061706).

Further, after much trial and error to study an apparatus for producing organic nanotubes, the inventors of the present invention found that a device suitable as means for dissolving a raw material in an organic solvent is the one that causes a suspension solution carried under pressure by a pump to pass through a very narrow orifice so that a high shearing force is applied to the suspension solution, whereby the solute is completely dissolved in a short time. Still further, the use of this device enables the amount of solvent required for the production of organic nanotubes to be significantly reduced to ⅕ to 1/10 of the amount required for the methods for producing organic nanotubes in the atmosphere in the aforementioned patent applications (PCT/JP2007/061703 and PCT/JP2007/061706).

In addition, an obtained emulsion requires filtration. This emulsion, which is a highly-concentrated dispersion liquid of organic nanotubes, requires much time for filtering and drying, and the emulsion may be changed in structure therein into a substance other than nanotubes. Therefore, there is a demand for means being capable of continuous drying with a high degree of efficiency. As a result of a study of the filtering and drying means, the inventors of the present invention found that a spray drier is suitable. The spray drier is a device that sprays a solution from a nozzle and performs continuous drying.

An object of the present invention is to provide a method and apparatus capable of continuously producing organic nanotubes, wherein an organic nanotube material dispersion solution consisting of an organic nanotube material and an organic solvent is pressurized and caused to pass through a very narrow orifice.

Another object of the present invention is to provide an apparatus capable of continuously producing organic nanotubes and realizing excellent drying efficiency, using a spray drier as drying means.

### Solution to Problem

In order to achieve the above object, a method for producing organic nanotubes according to the present invention comprises: pressurizing an organic nanotube material dispersion solution and causing the organic nanotube material dispersion solution to pass through an orifice, the organic nanotube material dispersion solution consisting of an organic nanotube material and an organic solvent; under an action of a shearing force caused when the organic nanotube material dispersion solution passes through the orifice, generating an excessively supersaturated solution in which the organic nanotube material is completely dissolved in the organic solvent; and cooling the excessively supersaturated solution, thereby forming an organic nanotube dispersion solution.

Further, a method for producing organic nanotubes according to the present invention comprises: spraying an organic nanotube dispersion solution from a spray nozzle of a spray drier device to separate the organic nanotube dispersion solution into a solvent vapor and organic nanotubes, thereby collecting the organic nanotubes in dry powder form.

Still further, an apparatus for producing organic nanotubes according to the present invention, comprises: a tank that contains an organic nanotube material dispersion solution consisting of an organic nanotube material and an organic solvent; a pump that pressurizes the organic nanotube material dispersion solution from the tank so that the organic nanotube material dispersion solution is carried under high pressure; a cylindrical casing for continuously flowing the organic nanotube material dispersion solution carried from the pump under pressure; an orifice being placed in the cylindrical casing; an organic nanotube precipitation pipe being coupled to an outlet of the cylindrical casing; and cooling means that cools the organic nanotube precipitation pipe to cause precipitation of organic nanotubes.

Yet further, an apparatus for producing organic nanotubes according to the present invention is such that the organic nanotube precipitation pipe is coupled to a spray nozzle of a spray drier device, and the spray drier device includes a drying chamber for spray-drying an organic nanotube dispersion solution sprayed from the spray nozzle, and the apparatus further comprises: dry air supplying means for supplying dry air to surroundings of the spray nozzle; and pressurized air supplying means for supplying pressurized air to the spray nozzle.

Further, an apparatus for producing organic nanotubes according to the present invention further comprises: a cyclone being connected via a carrying pipe to the drying chamber of the spray drier device; and a product container being provided below the cyclone, wherein an exhaust pipe being connected to an upper part of the cyclone is connected to an exhaust fan via a heat exchanger being provided around the organic nanotube precipitation pipe, and the exhaust fan is connected to a solvent recovery container.

Still further, an apparatus for producing organic nanotubes according to the present invention further comprises: a mesh being connected via a carrying pipe to the drying chamber of the spray drier device, wherein an exhaust pipe being provided around the mesh is connected to an exhaust fan via a heat exchanger being provided around the organic nanotube precipitation pipe, and the exhaust fan is connected to a solvent recovery container.

### ADVANTAGEOUS EFFECTS OF INVENTION

The method for producing organic nanotubes according to the present invention yields the following excellent effects.

(1) Adoption of means that pressurizes the organic nanotube material dispersion solution and causes organic nanotube material dispersion solution to pass through the orifice makes it possible to continuously synthesize and produce organic nanotubes. Further, it is possible to significantly reduce the required amount of solvent to ⅕ to 1/10 of the amount required in a method for producing organic nanotubes in the atmosphere.

(2) With the use of a spray drier device, it is possible to easily remove a solvent that is held strongly in a small inner space of the organic nanotube. Further, it is possible to perform drying in a short time, thus preventing the occurrence of thermal change in structure of the organic nanotube.

The apparatus for producing organic nanotubes according to the present invention yields the following excellent effects.

(1) With such an arrangement that there are provided: a pump that pressurizes the organic nanotube material dispersion solution so that the organic nanotube material dispersion solution is carried under high pressure; a cylindrical casing for continuously flowing the organic nanotube material dispersion solution carried from the pump under pressure; and an orifice being placed in the cylindrical casing, and an organic nanotube precipitation pipe being provided at an outlet of the cylindrical casing is coupled to a spray drier device, it is possible to provide an apparatus capable of continuously producing organic nanotubes and realizing excellent drying efficiency.

(2) With such an arrangement that there are provided: a pump that pressurizes the organic nanotube material dispersion solution so that the organic nanotube material dispersion solution is carried under high pressure; a cylindrical casing for continuously flowing the organic nanotube material dispersion solution carried from the pump under pressure; an orifice being placed in the cylindrical casing, and an organic nanotube precipitation pipe being provided at an outlet of the cylindrical casing is coupled to a spray drier device, a cyclone is connected to a drying chamber of the spray device, a product container is provided below the cyclone, an exhaust pipe being connected to an upper part of the cyclone is connected to an exhaust fan via a heat exchanger being provided around the organic nanotube precipitation pipe, and the exhaust fan is connected to a solvent recovery container, pressurization and cooling of the solution and recovery of the solvent, which are complicated operations that consumes a large amount of energy, can be realized by a simple apparatus that contributes to energy cost reduction. This can be also realized by the use of a mesh, instead of the cyclone.

### BRIEF DESCRIPTION OF DRAWINGS

FIG. 1 is an explanatory view generally showing an embodiment of a method and apparatus for producing organic nanotubes according to the present invention.

### REFERENCE SIGNS LIST

1 Tank
2 Pump
3 Cylindrical casing
4 Organic nanotube
5 Orifice
6 Organic nanotube precipitation pipe
7 Switch valve
8 Return pipe
10 Spray drier device
11 Spray nozzle
12 Drying chamber
13 Lower chamber
14 Dry air supplying means
15 Heater
16 Pressurized air supplying means
17 Carrying pipe
18 Cyclone
19 Product container
20 Exhaust pipe
21 Heat exchanger
22 Exhaust fan

### DESCRIPTION OF EMBODIMENTS

An organic nanotube material is dispersed in methanol, and an obtained solution is caused to pass through an orifice inside a cylindrical casing. This causes application of a high shearing force accompanied by a rise in temperature of the solution, thus instantaneously dissolving the organic nanotube material to form an excessively supersaturated solution. A pressure applied at the passage of the solution through the orifice is preferably as high as possible. For general devices, the pressure is 245 MPa at the maximum. After the passage of the solution through the orifice, the solution is cooled as quickly as possible by a heat exchanger. This causes self-assembling of the organic nanotube material in the excessively supersaturated solution, thus producing an organic nanotube dispersion solution.

Next, the organic nanotube dispersion solution is put through a spray drier device. The organic nanotube dispersion solution as well as dry air heated at a temperature equal to or less than a gel-liquid crystal transition temperature of the organic nanotube material is sprayed from a spray nozzle. This makes it possible to vaporize the solvent without destruction of the structure of an organic nanotube. Thereafter, the organic nanotube dispersion solution is continuously put through a cyclone so as to be separated into a solvent vapor and organic nanotubes. This makes it possible to produce organic nanotubes in dry powder form. Note that the organic nanotube dispersion solution may be produced by other method.

The following will describe details of an embodiment of production of organic nanotubes according to the present invention. However, the present invention is not limited to the embodiment and is susceptible of various changes, modifications, and improvements within the scope of the present invention on the basis of knowledge of a person skilled in the art.

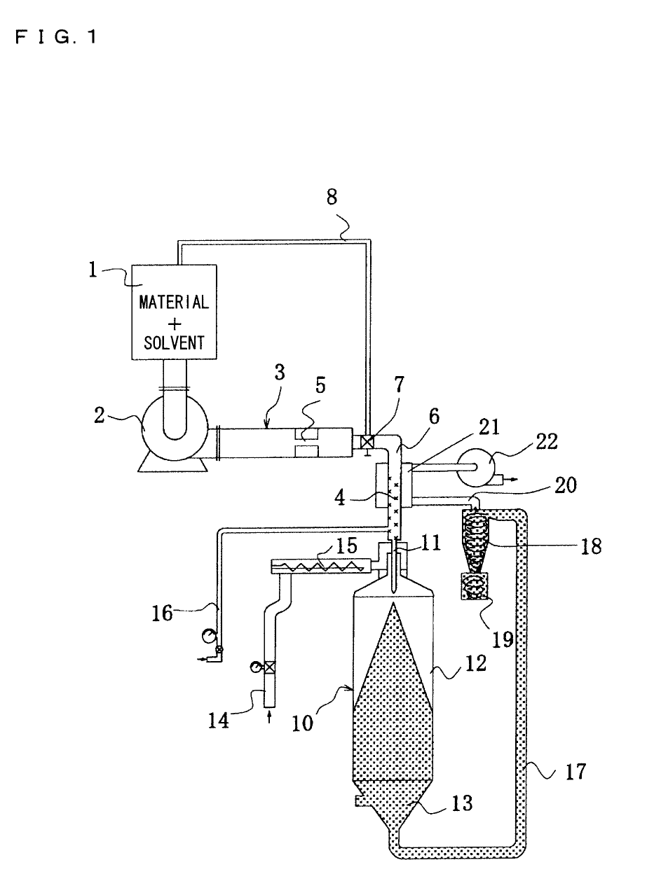

FIG. 1 is an explanatory view generally showing an embodiment of a method and apparatus for producing organic nanotubes according to the present invention. An organic nanotube producing apparatus is provided with a tank 1. In the tank 1, a material for organic nanotubes and an organic solvent are contained in predetermined amounts.

As the material for organic nanotubes, an N-glycoside type glycolipid or a peptide-lipid conjugate is used.

The N-glycoside type glycolipid is represented by the following general formula (1):

G-NHCO—R¹ (1)

wherein G represents a sugar residue resulting from removal of a hemiacetal hydroxyl group binding to an anomeric carbon atom from a sugar, and R1 represents an unsaturated hydrocarbon group having 10 to 39 carbon atoms.

The peptide-lipid conjugate is a peptide-lipid conjugate having a long-chain hydrocarbon group, which is represented by the following general formula (2):

R²CO(NH—CHR³—CO)ₘOH (2), or

the following general formula (3):

H(NH—CHR³Y—CO)ₘNHR² (3)

wherein R² represents a hydrocarbon group having 6 to 18 carbon atoms, R³ represents an amino acid side chain, and m represents an integer of 1 to 10.

A distinctive feature is to use an organic solvent as a solvent. This organic solvent is heated up to a temperature equal to or less than a boiling point thereof. This makes it possible to increase the amount of N-glycoside type glycolipid or peptide-lipid conjugate dissolved therein. A higher concentration of the N-glycoside type glycolipid or the peptide-lipid conjugate in the solution is preferable, a saturated state is most preferable.

The organic solvent to be used may be an alcohol-based solvent or a cyclic ether-based solvent, each having a boiling point of 120° C. or less. This organic solvent may be a single type of solvent or may be a mixture of two or more types of solvents. Further, this organic solvent may be mixed with a small amount of water.

In the tank 1, the organic nanotube material is partially solved or dispersed in the organic solvent so as to be prepared as a dispersion solution.

The tank 1 is connected to an inlet of a pump 2. An outlet of the pump 2 is connected to a cylindrical casing for continuously flowing the organic nanotube material dispersion solution discharged from the pump 2. In the cylindrical casing 3, a very narrow orifice 5 is provided for squeezing the flow of the organic nanotube material dispersion solution. In FIG. 1, the orifice 5 has a single step. Alternatively, the orifice 5 may have two or more steps. The organic nanotube material dispersion solution discharged from the pump 2 passes through the very narrow orifice 5 while being subjected to a high shearing force through a space lessened between wall surfaces of the orifice 5. This causes the solute to be dispersed in such a state that individual molecules of the solute are separated from each other (complete dissolution). The organic nanotube material dispersion solution passes through the orifice 5 under a pressure of approximately 100 MPa to 300 MPa.

An outlet of the cylindrical casing 3 is connected to an organic nanotube precipitation pipe 6. The organic nanotube precipitation pipe 6 is coupled to a spray nozzle 11 of a spray drier device 10 that serves as drying means. Further, the organic nanotube precipitation pipe 6 is connected to a return pipe 8 at a midpoint thereof. The return pipe 8 couples the organic nanotube precipitation pipe 6 to the tank 1. At the connection between the organic nanotube precipitation pipe 6 and the return pipe 8, a switch valve 7 is provided. With this arrangement, in the event when an organic nanotube precipitation failure occurs for some reason, the switch valve 7 is manipulated so that the organic nanotube material dispersion solution can be returned to the tank 1.

Complete dissolution of the organic nanotube material in the organic solvent with use of a conventional container requires a predetermined time and a predetermined amount of solvent. On the contrary, the organic nanotube material dispersion solution discharged from the pump 2 is subjected to a high shearing force accompanied by a rise in temperature of the solution when passing through the orifice 5. This realizes instantaneous and complete dissolution of the organic nanotube material in the solvent the amount of which is ⅕ to 1/10 of the conventionally required amount, which results in an excessively supersaturated solution in the organic nanotube precipitation pipe 6. In a process of cooling the excessively supersaturated solution in the organic nanotube precipitation pipe 6 by means of a heat exchanger 21 (which will be described later), self-assembling of the organic nanotube material occurs. This results in the precipitation of organic nanotubes 4.

The spray drier device 10 includes a drying chamber 12 for spray-drying a dispersion liquid containing the organic nanotubes 4 from the spray nozzle 11. A top part of the drying chamber 12 is connected to dry air supplying means 14 for supplying dry air to surroundings of the spray nozzle 11. A heater 15 is provided at a midpoint of the dry air supplying means 14.

Further, the spray nozzle 11 is connected to pressurized air supplying means 16 for supplying pressurized air to the spray nozzle 11.

The spray drier device 10 includes a lower chamber 13 that is provided below the drying chamber 12. The lower chamber 13 is connected via a carrying pipe 17 to a cyclone 18.

As to the sprayed dispersion liquid containing the organic nanotubes 4 from the spray nozzle 11, droplets are instantaneously evaporated in the drying chamber 12 so that organic nanotubes are formed in a dried state. Gas generated by the evaporation and the organic nanotubes in the dried state are all carried from the lower chamber 13 to the cyclone 18 via the carrying pipe 17.

Below the cyclone 18, a product container 19 is provided. An upper part of the cyclone 18 is connected to an exhaust pipe 20. The exhaust pipe 20 is connected to an exhaust fan 22 via the heat exchanger 21 for cooling the organic nanotube precipitation pipe 6. The exhaust fan 22 is provided at the end of the exhaust pipe 20. The heat exchanger 21 is set at temperatures in the range from −20° C. to 20° C.

The mixture of a vaporized organic solvent and the organic nanotubes is centrifuged by the cyclone 18. As a result, the organic nanotubes are collected in the product container 19, and the solvent vapor is subjected to heat exchange in the heat exchanger 21 and then carried via the exhaust fan 22 to a solvent recovery container (not shown). The solvent in a liquid state in the solvent recovery container is filled into the above-described tank 1 and used again in the production of organic nanotubes. Optionally, a filter for collecting ultra fine powder having unfortunately passed through the cyclone 18 may be placed in front of the exhaust fan 22.

Further, a device for separating the mixture of the vaporized organic solvent and the organic nanotubes, which is not limited to a cyclone, may be a mesh filter such as a bug filter, for example. In this case, the filter is surrounded by one size larger cylindrical container, so that the vaporized organic solvent is carried to the heat exchanger 21.

### Production Example 1

As the organic nanotube material, 25 g of the peptide-lipid conjugate represented by the general formula (2), wherein R² represents a hydrocarbon group having 13 carbon atoms, R³ represents hydrogen, and m represents an integer of 2, was dispersed in 1 liter of methanol. The resultant dispersion liquid was caused to pass through the orifice at a pressure of 245 MPa and at a flow rate of 300 ml per minute. Thereafter, the dispersion liquid was quickly cooled by the heat exchanger in which cold water of 5° C. was flown, so that a methanol dispersion liquid with organic nanotubes was produced.

### Production Example 2

As the organic nanotube material, 50 g of the peptide-lipid conjugate represented by the general formula (2), wherein R² represents a hydrocarbon group having 13 carbon atoms, R³ represents hydrogen, and m represents an integer of 2, was dispersed in 1 liter of methanol. The resultant dispersion liquid was heated to 50° C. and then caused to pass through the orifice at a pressure of 245 MPa and at a flow rate of 300 ml per minute. Thereafter, the dispersion liquid was quickly cooled by the heat exchanger inside which cold water of 5° C. was flown, so that a methanol dispersion liquid with organic nanotubes was produced.

### Production Example 3

As the organic nanotube material, 20 g of the peptide-lipid conjugate represented by the general formula (2), wherein R² represents a hydrocarbon group having 13 carbon atoms, R³ represents hydrogen, and m represents an integer of 2, was dispersed in 1 liter of methanol. The resultant dispersion liquid was heated to 50° C. and then caused to pass through the orifice at a pressure of 245 MPa and at a flow rate of 100 ml per minute. Immediately after the passage of the dispersion liquid, the dispersion liquid was quickly cooled by the heat exchanger inside which water of 20° C. was flown, so that a methanol dispersion liquid with organic nanotubes was produced.

### Production Example 4

170 ml of the organic nanotube dispersion liquid produced in Production Example 3 was discharged from the spray nozzle through the application of pressurized air heated at 60° C. by the heater. Thereafter, the resultant product was collected by the cyclone. As a result, 1.9 g of organic nanotubes was obtained in dry powder form.

### Production Example 5

200 ml of the organic nanotube dispersion liquid produced in Production Example 3 was discharged from the spray nozzle through the application of pressurized air heated at 60° C. by the heater. Thereafter, the resultant product was collected by the bug filter. As a result, 2.6 g of organic nanotubes was obtained in dry powder form.

We claim:

1. A method for producing organic nanotubes, comprising:
pressurizing an organic nanotube material dispersion solution and causing the organic nanotube material dispersion solution to pass through an orifice, the organic nanotube material dispersion solution consisting of an organic nanotube material and an organic solvent;
under an action of a shearing force caused when the organic nanotube material dispersion solution passes through the orifice, generating an excessively supersaturated solution in which the organic nanotube material is completely dissolved in the organic solvent; and
cooling the excessively supersaturated solution to cause precipitation of organic nanotubes, thereby forming an organic nanotube dispersion solution.

2. A method for producing organic nanotubes, comprising:
spraying an organic nanotube dispersion solution from a spray nozzle of a spray drier device to separate the organic nanotube dispersion solution into a solvent vapor and organic nanotubes, thereby collecting the organic nanotubes in dry powder form.

3. An apparatus for producing organic nanotubes, comprising:
a tank that contains an organic nanotube material dispersion solution consisting of an organic nanotube material and an organic solvent;
a pump that pressurizes the organic nanotube material dispersion solution from the tank so that the organic nanotube material dispersion solution is carried under high pressure;
a cylindrical casing for continuously flowing the organic nanotube material dispersion solution carried from the pump under pressure;
an orifice being placed in the cylindrical casing;
an organic nanotube precipitation pipe being coupled to an outlet of the cylindrical casing; and
cooling means that cools the organic nanotube precipitation pipe to cause precipitation of organic nanotubes.

4. The apparatus according to claim 3, wherein
the organic nanotube precipitation pipe is coupled to a spray nozzle of a spray drier device, and the spray drier device includes a drying chamber for spray-drying an organic nanotube dispersion solution sprayed from the spray nozzle, and the apparatus further comprises: dry air supplying means for supplying dry air to surroundings of the spray nozzle; and pressurized air supplying means for supplying pressurized air to the spray nozzle.

5. The apparatus according to claim 4, further comprising:
a cyclone being connected via a carrying pipe to the drying chamber of the spray drier device; and
a product container being provided below the cyclone,
wherein an exhaust pipe being connected to an upper part of the cyclone is connected to an exhaust fan via a heat exchanger being provided around the organic nanotube precipitation pipe, and the exhaust fan is connected to a solvent recovery container.

6. The apparatus according to claim 4, further comprising:
a mesh being connected via a carrying pipe to the drying chamber of the spray drier device,
wherein an exhaust pipe being provided around the mesh is connected to an exhaust fan via a heat exchanger being provided around the organic nanotube precipitation pipe, and the exhaust fan is connected to a solvent recovery container.
